(19) United States
(12) Patent Application Publication
(10) Pub. No.: US 2024/0197774 A1
(43) Pub. Date: Jun. 20, 2024

(54) Synthesis of Antimicrobial PVP-coated Bismuth Nanoparticles

(71) Applicant: Board of Regents, The University of Texas System, Austin, TX (US)

(72) Inventors: JOSE LOPEZ-RIBOT, San Antonio, TX (US); ROBERTO VAZQUEZ-MUNOZ

(21) Appl. No.: 18/287,009

(22) PCT Filed: Apr. 16, 2022
(86) PCT No.: PCT/US2022/025146

§ 371 Date: Oct. 15, 2023

Related U.S. Application Data
(60) Provisional application No. 63/176,086, filed on Apr. 16, 2021.

(51) Int. Cl. A61K 33/245; A61K 9/51; B82Y 5/00; B82Y 40/00

(52) U.S. Cl. CPC A61K 33/245; A61K 9/5138; B82Y 5/00; B82Y 40/00

(57) ABSTRACT

Described herein is a facile, fast, and economical method for the synthesis of BAL-mediated PVP-BiNPs, using basic laboratory instruments and reagents readily available in most laboratories.

### RELATED APPLICATION

This Application is an international application claiming priority to U.S. Provisional Patent Application Ser. No. 63/176,086 filed on Apr. 16, 2021 which is incorporated herein by reference in its entirety.

### STATEMENT REGARDING FEDERALLY FUNDED RESEARCH

None.

### BACKGROUND

Bismuth (Bi, atomic number 83) is a water-insoluble metallic element used in a wide array of medical applications, because it is considered non-toxic for humans (lethal Intake>5-20 g/day/Kg for years)(Ma et al., J. Inorg. Biochem. 168: 38-45, 2017; Sun, et al., Chapter 1. The Chemistry of Arsenic, Antimony and Bismuth, Biological Chemistry of Arsenic, Antimony and Bismuth, 1st ed., Wiley, United Kingdom, 2011). When bismuth is chelated with hydroxyl or sulfhydryl containing molecules, its water solubility and biocompatibility are both increased. The water solubility and lipophilicity of bismuth are substantially enhanced when bismuth ions (Bi³⁺) are complexed with small lipophilic molecules, such as dimercaptopropanol (BAL)(Badireddy et al., J. Nanoparticle Res. 16:6, 2014). BAL is an FDA approved drug (FDA-Approved Drugs, U.S. Food and Drug Administration, 2019 URL www.accessdata.fda.gov/scripts/cder/daf/index.cfm?event-BasicSearch.process) for treatments of metal poisoning (Andersen, Mini Rev. Med. Chem. 4(1): 11-21, 2005).

Bismuth is used in the manufacture of pharmaceutical products, cosmetics, catalysts, pigments, electronics, and alloys. Water-soluble biocompatible bismuth complexes are used in health and cosmetics products, and medicine. Also, bismuth compounds present antimicrobial properties. It has been demonstrated that bismuth exhibits high antibacterial activities against several bacterial species, including Clostridium difficile, Helicobacter pylori, Escherichia coli, Pseudomonas aeruginosa, Proteus mirabilis, and Staphylococcus aureus (Folsom et al., J. Appl. Microbiol. 111(4):989-96, 2011; Mahony et al., Antimicrob. Agents Chemother 43(3): 582-88, 1999). BAL-bismuth compounds display increased antibacterial activity (Domenico, et al., Antimicrobial Agents and Chemotherapy 41(8): 1697-1703, 1997). Yet, there is a lack of investigation into methods for synthesizing antimicrobial nanoparticles (nanoantibiotics).

Bismuth-based nanostructures have been used for different applications, such as photocatalytic oxidative desulfurization processes (Mousavi-Kamazani, J. Mater. Sci. Mater. Electron. 30(19): 17735-40, 2019; Mousavi-Kamazani, J. Alloy. Compd. 823: 153786, 2020). There are only a handful of studies regarding the synthesis and evaluation of bismuth nanoparticles for antimicrobial treatments. Usually, the synthesis methods for BiNPs require specialized equipment (El-Batal et al., J. Photochem. Photobiol. B Biol. 173:120-39, 2017; Reus et al., Toxicol. In Vitro 53: 99-106, 2018) or controlled conditions (Bi et al., Chem. Mater. 30(10): 3301-07, 2018; Gomez et al., Ultrason. Sonochem. 56: 167-73, 2019; Wei et al., ACS Appl. Mater. Interfaces 8(20): 12720-26, 2016; Winter et al., Nanotechnology 29(15): 155603, 2018). As such, most current protocols for synthesizing biologically suitable BiNPs cannot be replicated in non-specialized laboratories without great difficulties (Badireddy et al., J. Nanoparticle Res. 16:6, 2014; Brown and Goforth, Chem. Mater. 24(9): 1599-1605, 2012; Petsom et al., Mater. Today Proc. 5(6): 14057-62, 2018). Facilitated protocols for nanomaterials synthesis expand the research for biomedical applications (Vazquez-Muñoz et al., BMC Res. Notes 12(1): 773, 2019). Described herein is a fast, facile, and economical method for synthesizing BiNPs that does not require the use of advanced equipment.

Thus, there remains a need for additional methods for synthesis of bismuth nanoparticles.

### SUMMARY

Bismuth is a water-insoluble non-toxic metallic element used in a wide array of pharmaceutical products, cosmetics, and catalysts. Yet, the research regarding the use of bismuth nanoparticles (BiNPs) for antimicrobial treatments is scarce. Most of the current protocols for synthesizing BiNPs suitable for medical uses cannot be easily replicated in non-specialized laboratories. The methods described herein provide a fast, facile, and economical methods for synthesizing BiNPs. Bismuth nanoparticles are synthesized by a chemical reduction process. In certain aspects, the BiNPs are synthesized in less than 1 h. The process can include synthesizing BiNPs in a heated alkaline glycine solution by the chelation and reduction of the bismuth ions using dimercaptopropanol (BAL) and sodium borohydride respectively, and then coated and stabilized by polyvinylpyrrolidone (PVP). The resulting PVP-BiNPs can be characterized by UV-Vis spectrophotometry and transmission electron microscopy (TEM). These nanoparticles can be potent nanoantibiotics.

Certain embodiments are directed to a method for synthesizing a coated bismuth nanoparticle (BiNP) comprising:

Adding Bi(NO₃)₃*5H₂O or other Bi salt to a solution or solubilizing Bi(NO₃)₃*5H₂O or other Bi salt at 60, 65, 70, 75, 80, 85, 90, to 95° C. and incubating for 1, 2, 3, 4, 5, 6, 7, 8, 9, 10, 11, 12, 13, 14, to 15 minutes forming a Bi(NO₃)₃ solution. In certain aspects Bi(NO₃)₃*5H₂O is added to a solution at 70 to 90° C. and incubating for 2 to 10 minutes forming a Bi(NO₃)₃ solution. In particular aspects the solution is a glycine, alanine, or similar solution.

Adding an inorganic base, for example a hydroxide of an alkali metal (e.g., LiOH, NaOH, KOH, etc) in an amount to raise the pH to at least 8.5, 9, 9.5 to 10 for 1, 2, 3, 4, 5, 6, 7, 8, 9, to 10 minutes. Adjusting the pH of the Bi(NO₃)₃ solution to at least 8.5, 9, 9.5 to 10. In certain aspects the pH is maintained at pH 8.5 to 10 throughout the process until the PVP-BiNPs are isolated.

Adding a chelator, e.g., dimercaptopropanol (BAL) Meso-2,3-dimercaptosuccinic acid (DMSA), sodium 2,3-dimercapto-1-propanesulfonate (DMPS), sodium 4,5-dihydroxybenzene-1,3-disulfonate (TIRON) and the like; and then polyvinyl pyrrolidone (e.g., PVP-10K) around a minute after adding the chelator. PVP is a water-soluble polymer made from the monomer N-vinylpyrrolidone. In certain aspects the reagents are added in this specific order, as BAL increases bismuth solubility, allowing it to be reduced and coated by the PVP. PVP of different molecular sizes (5K to 1300K including all sizes there between) may produce nanoparticles with similar properties. In certain aspects, PVP is PVP-10K. Also, other molecules may work too, however sodium citrate produced highly unstable nanoparticles. The chelator is added while stirring or mixing the solution and incubating for about 1 to 3 minutes forming a precipitation precursor solution. The length of incubation effects the size and shape of the nanoparticles produced.

Precipitating bismuth nanoparticles by adding a reducing agent, such as NaBH₄, dropwise until the color of the solution changes to a dark color, e.g., black (the dark color indicates formation of the nanoparticles) forming PVP coated BiNPs (PVP-BiNPs). In certain aspects the NaBH₄ is added over 1 to 10 minutes. The term “dropwise” refers to adding one solution to another discontinuously, intermittently, slowly, via discrete or separate aliquots. The aliquots can have a volume of or about 1 to 20 μL, more particularly 5 to 10 μL.

Optionally, adding another volume of NaBH₄ dropwise and mixing the solution for about 5 to 20 minutes.

The method further comprising washing the PVP-BiNPs. Washing can include isolating (e.g., pelleting, filtering or the like) the PVP-BiNPs, removing the supernatant, and washing the isolated PVP-BiNPs with water. In certain aspects, the PVP-BiNPs are isolated by centrifugation at 4000 rpm for 25 minutes (about 3500 to 5000 g) and supernatant removed.

In certain aspects, the isolated PVP-BiNPs are dried, forming a dry powder. The dried powder PVP-BiNPs can be stored under the appropriate conditions, such as a temperature of about 4° C. or lower. In certain aspects, the dried powder PVP-BiNPs are protected from light, such as being stored in a light safe container or location, e.g, storing the nanoparticles in the dark. In certain aspects the PVP-BiNPs can exhibit antimicrobial activity against bacteria and fungi; antibiofilm activity; and stability in solutions.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention.

Throughout this application, the term “about” is used to indicate that a value includes the inherent variation of error for the device, the method being employed to determine the value, or the variation that exists among the study subjects.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

As used herein, the term “IC₅₀” refers to an inhibitory dose that results in 50% of the maximum response obtained.

The term half maximal effective concentration (EC₅₀) refers to the concentration of a drug that presents a response halfway between the baseline and maximum after some specified exposure time.

The terms “inhibiting,” “reducing,” or “prevention,” or any variation of these terms, when used in the claims and/or the specification includes any measurable decrease or complete inhibition to achieve a desired result.

The use of the term “or” in the claims is used to mean “and/or” unless explicitly indicated to refer to alternatives only or the alternatives are mutually exclusive, although the disclosure supports a definition that refers to only alternatives and “and/or.”

As used herein, the term “patient” or “subject” refers to a living mammalian organism, such as a human, monkey, cow, sheep, goat, dogs, cat, mouse, rat, guinea pig, or species thereof. In certain embodiments, the patient or subject is a primate. Non-limiting examples of human subjects are adults, juveniles, infants and fetuses.

The use of the word “a” or “an” when used in conjunction with the term “comprising” in the claims and/or the specification may mean “one,” but it is also consistent with the meaning of “one or more,” “at least one,” and “one or more than one.”

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1D. Synthesis process: from the initial colorless bismuth salt solution (A), it turned to a turbid white color after the addition of NaOH and PVP (B); when BAL was added it changed to a translucent yellow color (C). Finally, when NaBH₄ is added, the solution immediately turned to pitch-black color (D).

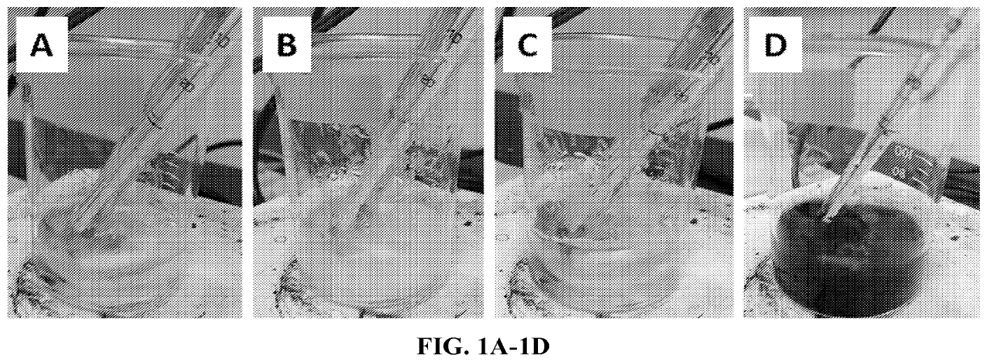

FIG. 2A-2C. Proposed mechanism of synthesis for the formation of BiNPs. Bismuth (III) ions, solubilized in a glycine solution (A), interact with BAL, leading to the formation of the bismuth-BAL complex (B). Finally, NaBH₄ induces the generation of PVP-BiNPs (C). Hydrogens atoms were not included in the molecular models for clarity. Molecules were built using the GLmol engine at URL molview.org/

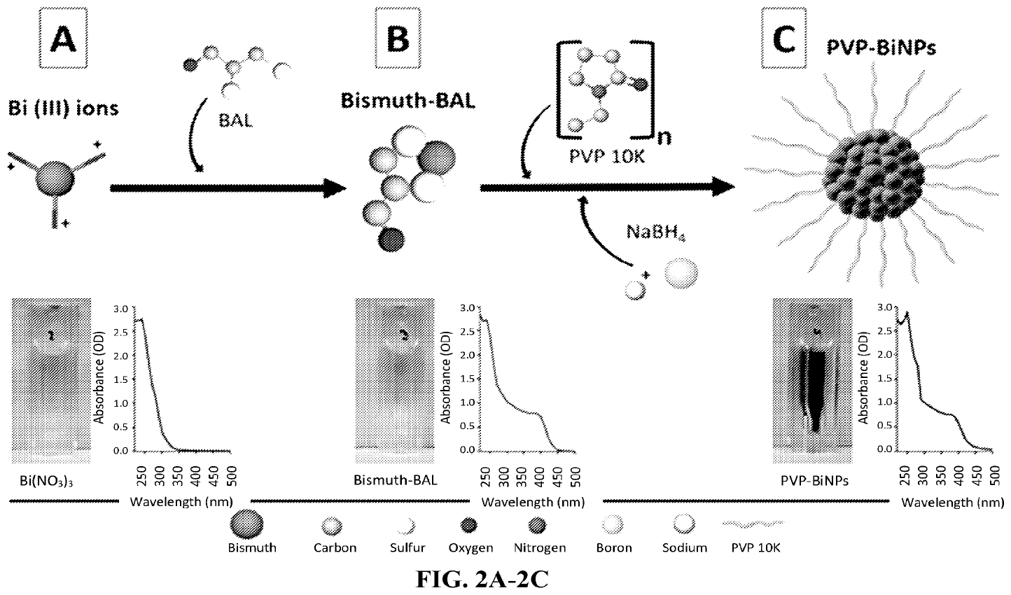

FIG. 3. The UV Vis absorbance profile of the BAL-mediated PVP-BiNPs reveals a peak around the 400 nm position.

FIG. 4A-4D. Electron microscopy characterization. HR-TEM images reveal that the BAL-mediated PVP-BiNPs were small nanoparticles, most of them with an aspect ratio close to 1 (A). The size distribution for the statistical analysis is shown in panel (B). An EDS Analysis confirms the presence of Bismuth on the nanoparticles (C). A closer view reveals the crystalline arrangement of the nanoparticles. Scale bar: 50 nm (A), 5 nm (D).

FIG. 5A-5B. (A) HR-TEM analysis from a single particle confirms their crystalline organization, whereas the (B) Electron Diffraction Pattern reveals their crystalline lattice as a cubic and hexagonal organization

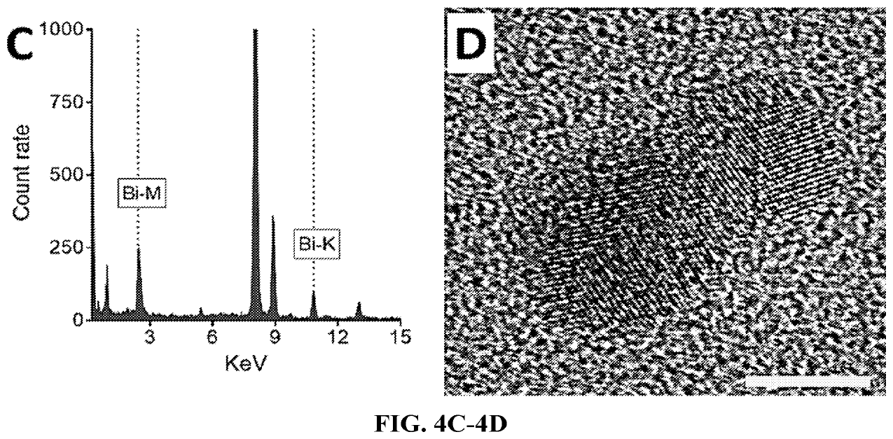

FIG. 6. The DLS analysis reveals that the bismuth nanoparticles size is around 20 nm, although some nanoparticles and clusters are larger than 100 nm.

### DESCRIPTION

Bismuth nanoparticles have antimicrobial properties, this easy-to-replicate protocol may further the research and use of bismuth nanoparticles for biomedical applications. The methods for the synthesis of bismuth nanoparticles described herein is simple, rapid, and inexpensive. The methods allow synthesis of small nanoparticles with an aspect ratio close to one. This multi-purpose nanotechnology provides a basis for disinfecting formulations. Certain embodiments are directed to disinfecting formulations for reducing and preventing the presence of cells and biofilms of disease- and food poisoning-associated pathogenic microorganisms. The formulations can be applied in hospitals, food courts/banks, and other facilities where is critical for reducing the number of microorganisms.

Synthesis of BiNPs. Reagents include bismuth nitrate [Bi(NO₃)₃*5H₂O], sodium borohydride (NaBH₄), 2,3-dimercapto-1-propanol (BAL), sodium hydroxide (NaOH), polyvinylpyrrolidone MW=10 KD (PVP-10 K), and glycine. All reagents can be purchased from Sigma Aldrich (MO). Equipment includes, but is not limited to 200 ml beaker, stirring hot plate, stir bar, thermometer, pH-meter, 12 ml plastic tubes, 1000 μl pipette, 200 μl pipette, 50 mL plastic tube, aluminum foil.

Methods of Preparation. It is preferable that the beaker, thermometer, pH-meter, and the stir bar are clean and washed with distilled water. Stock solutions can be prepared using Milli Q water (or distilled water), the stock solutions can include 1 M glycine, 3 M NaOH, 3 mM PVP, and 1 M NaBH₄. NaBH₄ can lose its activity very fast when diluted in water, it should be freshly prepared, immediately before using it. PVP-10 K molecular mass is 10,000. For a 3 mM solution, 0.3 g of PVP were diluted in 10 ml of Milli Q water. For a typical reaction, low volumes can be used: as an example, for a single-synthesis reaction (in Milli Q water or distilled water) the following were prepared: 1 M glycine (20 ml), 3 M NaOH (5 ml), 3 mM PVP (5 ml), and 1 M NaBH₄ (10 ml). These solutions can be scaled up and modified for large scale production of the BiNPs. Bismuth nitrate and BAL are used as received. BAL has a strong odor, and it should be opened and handled inside a chemical hood. The use of gloves and masks, and all other appropriate safety measures and pertinent protective equipment, is highly encouraged during the synthesis process.

Synthesis of the PVP-BiNPs. In one example of the synthetic methods BAL-mediated PVP-BiNPs were synthesized by the chemical reduction of bismuth ions in an organic solution. The following is a step-by-step description of the proposed synthesis method. 20 ml of 1 M glycine solution were heated to 70±5° C. to form a suspension, under continuous vigorous stirring. This temperature is maintained through the synthesis process. The temperature is needed for the proper synthesis. Lower temperatures (e.g., less than 65° C.)result in a highly unstable suspension that precipitates within minutes after the synthesis. (i) 146.2 μg of the Bi(NO₃)₃*5 H₂O crystals were added to the pre-warmed glycine solution (for an initial 15 mM bismuth solution). (ii) After approximately 2 min, enough volume of 3 M NaOH was added to raise the solution pH to 9. This turns the solution from transparent to a turbid white color. An alkaline pH is kept during the whole synthesis process. pH is likely to drop during the synthesis process, more NaOH can be added to keep it at about 9 or above. (iii) After 3 min, 75 μL of 8.1 M of 2,3-dimercaptopropanol (BAL) were added, rapidly turning the turbid whitish appearance to a translucent bright yellow color. Immediately, 3 mL of 3 mM PVP-K10 were added to the stirring suspension. (iv) About 1 min later, 5 mL of 1 M NaBH₄ were added dropwise. The suspension rapidly turns to a deep black color. NaBH₄ induces an exothermic reaction increasing the temperature of the solution, it is needed to be added slowly for safety reasons. Approximately 3 min later, another 2 ml of 1 M NaBH₄ were added dropwise and it was left for vigorous stirring for about 10 additional minutes. If the protocol is followed, and the volume of NaOH was around 1.5-2 ml, the final concentration of the total bismuth should be around about 9.3-9.5 mM (about 1254-1985 μg/ml). There may be other bismuth species in the solution but those can be removed by washing the BiNPs (see below). The BAL-mediated PVP-BiNPs black suspension can be stored in a Falcon® plastic tube and cooled down to room temperature and posteriorly stored at 4° C.

Optionally, the Bismuth nanoparticles can be washed to remove other bismuth species. An example of a washing process can include: pelleting BiNPs by centrifugation at 4000 rpm for 25 min and then washed with Milli-Q water, twice. BiNPs can be centrifuged again, then left to dry until they form a dry powder, then kept at 4° C., in a light-protected container.

Dried BiNPs can be suspended in sterile Milli Q water. If BiNPs are washed, the concentration of bismuth can be adjusted as desired, by weighting the BiNPs in the desired volume of Milli Q water.

Validation. After the method was standardized, the BAL-mediated BiNPs were synthesized in more than 10 rounds, on different days, to verify the reproducibility of the protocol. The measurement of the BiNPs size was performed on randomly selected different rounds of synthesis, for the TEM and the DLS analysis. The statistical analysis was performed on the Prism 8 (GraphPad Software Inc) software.

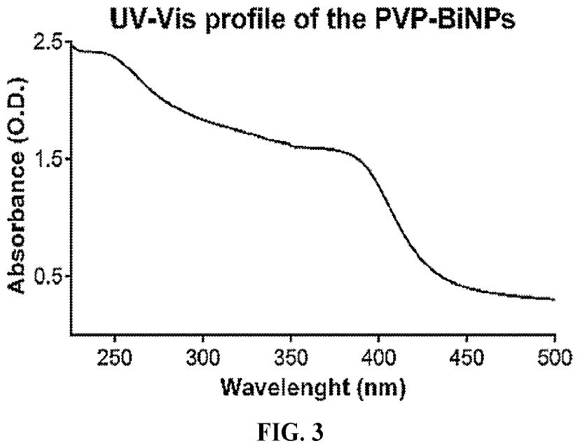

Characterization of the BAL mediated PVP-BINPs UV-Vis spectroscopy. BAL-mediated PVP-BiNPs absorbance profile was collected in a UV-Vis-NIR Cary 500 spectrophotometer (Agilent Technologies), in a wavelength range from 225 to 500 nm, in 1 nm steps. Bismuth Nanoparticles showed a constant absorbance from 225 nm to 500 nm, then decreases at 2=385 nm. Results from the UV-Vis spectrophotometry analysis suggest the transformation from bismuth (III) ions to bismuth nanoparticles (FIG. 3). This UV-Vis profile is similar to the one reported by Wang et al. for PVP-BiNPs (Wang et al., J. Phys. Chem. 109(15): 7067-72, 2005).

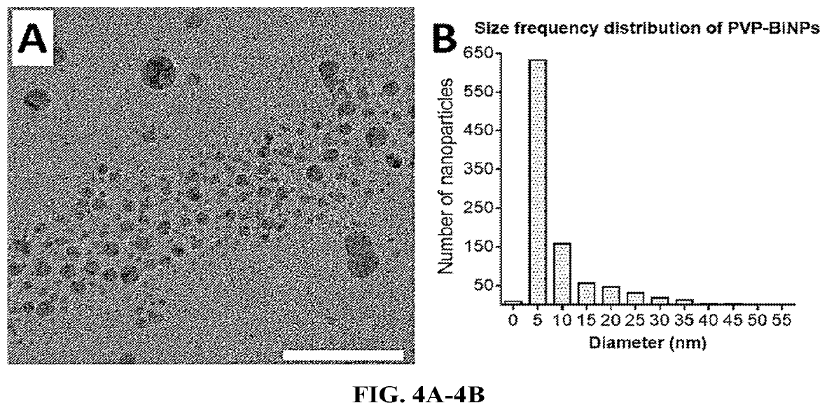

High-resolution transmission electron microscopy. 10 μL from the PVP-BINPs suspension were deposited on Type-B Carbon-coated copper grids (Ted Pella Inc.) and left to dry overnight. The BiNPs were analyzed in a JEOL 2010-F HR-TEM (Jeol Ltd.), with an accelerating voltage of 200 kV. TEM images confirm the presence of small nanoparticles, with an aspect ratio of close to 1, e.g., aspect ratio of 1, 1.25, 1.5, 1.75 to 2 being attainable (FIG. 4A). The statistical analysis of the frequency distribution size (performed on Prism 8, GraphPad Software Inc.) reveals that the average diameter of the nanoparticles was 8.57±7.52 nm (n=964) (FIG. 4B). An Energy Dispersive X-ray spectroscopy (EDS) analysis was performed to assess the chemical elemental composition of the nanoparticles. The AgNPs were deposit in a copper grid (Ted Pella) and analyzed using an EDAX collector (FIG. 4C). The HR-TEM analysis of a single particle confirms the crystalline arrangement of the bismuth nanoparticles (FIG. 4D).

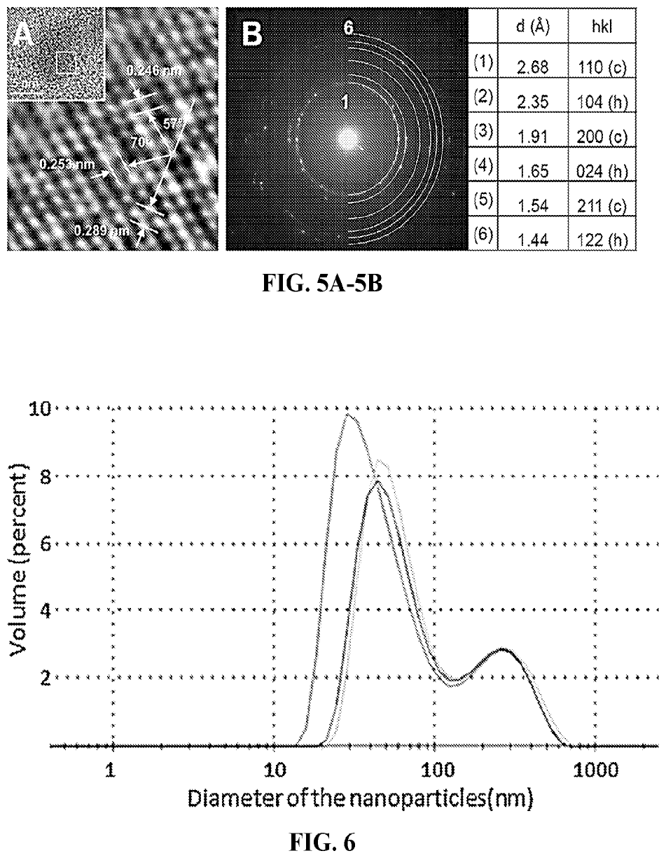

Dynamic light scattering (DIS) analysis. The Hydrodynamic size of the BAL-mediated PVP-BiNPs was determined by a DLS analysis. Briefly, the bismuth nanoparticles—diluted in Milli Q water—were transferred to a DTS1070 cell and analyzed in a Zetasizer Nano ZS (Malvern Panalytical), at room temperature, in triplicate. The BAL-mediated PVP-BINPs hydrodynamic size is 22.5±0.06 (FIG. 6). The hydrodynamic size is greater than the metallic core observed on electron micrographs. This can be attributed to the extended PVP chain-like molecules from the coating, which hydrated under the aqueous environment. According to Wang et al., FTIR demonstrates that PVP interacts chemically with bismuth (Wang et al., J. Phys. Chem. 109(15): 7067-72, 2005), resulting in PVP-coated bismuth nanoparticles.

Described herein is a facile, fast, and economical method for the synthesis of BAL-mediated PVP-BiNPs, using basic laboratory instruments and reagents readily available in most laboratories. These BAL-mediated PVP-BiNPs are small spheroids (<15 nm).

The properties of the compositions of the present invention are desirable in a broad field of applications. They are suitable for use in the medical field, particularly where there is a high risk of contamination and infection. In addition to first responders such as ambulance, law enforcement and fire personnel, this invention can have application in the dental profession.

In addition to being a disinfectant, embodiments of this invention also act as a sanitizer and a cleanser. The invention has application to the beauty industry, particularly as an additive to some cosmetics. Some formulations of the invention be used as a percentage addition to creams and ointments as an anti-microbial component or as a component for single or multiple-use wipes.

Bismuth-based nanocomposites, which can be used alone or in combination with benzalkonium chloride, tetrabutylammonium chloride, alcohols, chlorine-based compounds, formaldehyde, glutaraldehyde, hydrogen peroxide, iodophors, ortho-phthalaldehyde (OPA), peracetic acid, and other chemical disinfectants.

### I. Methods of Treatment

The compositions of the invention can be used to treat a number of microbial infections.

Bacterial Pathogens. The bacterium can be a Gram-positive bacterium or a Gram-negative bacterium. Bacterial pathogens include, but are not limited to, Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli O157:H7, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Preteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, and Yersinia pestis.

Bacterial pathogens may also include bacteria that cause resistant bacterial infections, for example, clindamycin-resistant Clostridium difficile, fluoroquinolon-resistant Clostridium difficile, methicillin-resistant Staphylococcus aureus (MRSA), multidrug-resistant Enterococcus faecalis, multidrug-resistant Enterococcus faecium, multidrug-resistance Pseudomonas aeruginosa, multidrug-resistant. Acinetobacter baumannii, and vancomycin-resistant Staphylococcus aureus (VRSA).

Antibiotic Combinations. In one embodiment, the compositions and methods of the present invention may be administered in conjunction with one or more antibiotics or antibacterial agents.

Anti-bacterial agents include, but are not limited to, aminoglycosides (e.g., amikacin (KANTREX®), neomycin (AMIKIN®), gentamicin (GARAMYCIN®), kanamycin (MYCIFRADIN®), netilmicin (NETROMYCIN®), tobramycin (NEBCIN®), Paromomycin (HUMATIN®)), ansamycins (e.g., geldanamycin, herbimycin), carbacephem (e.g., loracarbef (LORABID®), Carbapenems (e.g., ertapenem (INVANZ®), doripenem (DORIBAX®), imipenem/cilastatin (PRIMAXIN®), meropenem (MERREM®), cephalosporins (first generation) (e.g., cefadroxil (DURICEF®), cefazolin (ANCEF®), cefalotin or cefalothin (KEFLIN®), cefalexin (KEFLEX®), cephalosporins (second generation) (e.g., cefaclor (CECLOR®), cefamandole (MANDOL®), cefoxitin (MEFOXIN®), cefprozil (CEFZIL®), cefuroxime (CEFTIN®, ZINNAT®)), cephalosporins (third generation) (e.g., cefixime (SUPRAX®), cefdinir (OMNICEF®, CEFDIEL®), cefditoren (SPECTRACEF®), cefoperazone (CEFOBID®), cefotaxime (CLAFORAN®), cefpodoxime (VANTIN®), ceftazidime (FORTAZ®), ceftibuten (CEDAX®), ceftizoxime (CEFIZOX®), ceftriaxone (ROCEPHIN®)), cephalosporins (fourth generation) (e.g., cefepime (MAXIPIME®)), cephalosporins (fifth generation) (e.g., ceftobiprole (ZEFTERA®)), glycopeptides (e.g., teicoplanin (TARGOCID®), vancomycin (VANCOCIN®), telavancin (VIBATIV®)), lincosamides (e.g., clindamycin (CLEOCIN®), lincomycin (LINCOCIN®)), lipopeptide (e.g., daptomycin (CUBICIN®)), macrolides (e.g., azithromycin (ZITHROMAX®, SUMAMED®, ZITROCIN®), clarithromycin (BIAXIN®), dirithromycin (DYNABAC®), erythromycin (ERYTHOCIN®, ERYTHROPED®), roxithromycin, troleandomycin (TAO®), telithromycin (KETEK®), spectinomycin (TROBICIN®)), monobactams (e.g., aztreonam (AZACTAM®)), nitrofurans (e.g., furazolidone (FUROXONE®), nitmfurantoin (MACRODANTIN®, MACROBID®)), penicillins (e.g., amoxicillin (NOVAMOX®, AMOXIL®), ampicillin (PRINCIPEN®), azlocillin, carbenicillin (GEOCILLIN®), cloxacillin (TEGOPEN®), dicloxacillin (DYNAPEN®), flucloxacillin (FLOXAPEN®), mezlocillin (MEZLIN®), methicillin (STAPHCILLIN®), nafcillin (UNIPEN®), oxacillin (PROSTAPHLIN®), penicillin G (PENTIDS®), penicillin V (PEN-VEE-KR), piperacillin (PIPRACIL®), temocillin (NEGABAN®), ticarcillin (TICAR®)), penicillin combinations (e.g., amoxicillin/clavulanate (AUGMENTIN®), ampicillin/sulbactam (UNASYN®), piperacillin/tazobactam (ZOSYN®), ticarcillin/clavulanate (TIMENTIN®)), polypeptides (e.g., bacitracin, colistin (COLY-MYCIN-S®), polymyxin B, quinolones (e.g., ciprofloxacin (CIPRO®, CIPROXIN®, CIPROBAY®), enoxacin (PENETREX®), gatifloxacin (TEQUIN®), levofloxacin (LEVAQUIN®), lomefloxacin (MAXAQUIN), moxifloxacin (AVELOX®), nalidixic acid (NEGGRAM®), norfloxacin (NOROXIN®), ofloxacin (FLOXIN®, OCUFLOX®), trovafloxacin (TROVAN®), grepafloxacin (RAXAR®), sparfloxacin (ZAGAM®), temafloxacin (OMNIFLOX®)), sulfonamides (e.g., mafenide (SULFAMYLON®), sulfonamidochrysoidine (PRONTOSIL®), sulfacetamide (SULAMYD®, BLEPH-10®), sulfadiazine (MICRO-SULFON®), silver sulfadiazine (SILVADENE®), sulfamethizole (THIOSULFIL FORTE®), sulfamethoxazole (GANTANOL®), sulfanilimide, sulfasalazine (AZULFIDINE®), sulfisoxazole (GANTRISIN®), trimethoprim (PROLOPRIM®), TRIMPEX®), trimethoprim-sulfamethoxazole (co-trimoxazole) (TMP-SMX) (BACTRIM®, SEPTRA®)), tetracyclines (e.g., demeclocycline (DECLOMYCIN®), doxycycline (VIBRAMYCIN®), minocycline (MINOCIN®), oxytetracycline (TERRAMYCIN®), tetracycline (SUMYCIN®, ACHROMYCIN® V, STECLIN®)), drugs against mycobacteria (e.g., clofazimine (LAMPRENE®), dapsone (AVLOSULFON®), capreomycin (CAPASTAT®), cycloserine (SEROMYCIN®), ethambutol (MYAMBUTOL®), ethionamide (TRECATOR), isoniazid (I.N.H. ®), pyrazinamide (ALDINAMIDE®), rifampin (RIFADIN®, RIMACTANE®), rifabutin (MYCOBUTIN®), rifapentine (PRIFTIN®), streptomycin), and others (e.g., arsphenamine (SALVARSAN®), chloramphenicol (CHLOROMYCETIN®), fosfomycin (MONUROL®), fusidic acid (FUCIDIN®), linezolid (ZYVOX®), metronidazole (FLAGYL®), mupirocin (BACTROBAN®), platensimycin, quinupristin/dalfopristin (SYNERCID®), rifaximin (XIFAXAN®), thiamphenicol, tigecycline (TIGACYL®), tinidazole (TINDAMAX® FASIGYN®)).

### II. Formulations

In view of the current specification, the determination of an appropriate treatment regimen (e.g., dosage, frequency of administration, systemic vs. local, etc.) is within the skill of the art. For administration, the components described herein will be formulated in a unit dosage form (solution, suspension, emulsion, etc.) in association with a pharmaceutically acceptable carrier. Such carrier vehicles are usually nontoxic and non-therapeutic. Examples of such vehicles are water, saline, Ringer's solution, dextrose solution, and Hank's solution. Non-aqueous vehicles such as fixed oils and ethyl oleate may also be used. A preferred vehicle is 5% (w/w) human albumin in saline. The vehicle may contain minor amounts of additives, such as substances that enhance isotonicity and chemical stability, e.g., buffers and preservatives.

The therapeutic compositions described herein, as well as their biological equivalents, can be administered independently or in combination by any suitable route. Examples of parenteral administration include intravenous, intraarterial, intramuscular, intraperitoneal, and the like. The routes of administration described herein are merely an example and in no way limiting.

The dose of the therapeutic compositions administered to an animal, particularly in a human, in accordance with embodiments of the invention, should be sufficient to result in a desired response in the subject over a reasonable time frame. It is known that the dosage of therapeutic compositions depends upon a variety of factors, including the strength of the particular therapeutic composition employed, the age, species, condition or disease state, and the body weight of the animal.

Moreover, dose and dosage regimen, will depend mainly on the type of biological damage to the host, the type of subject, the history of the subject, and the type of therapeutic composition being administered. The size of the dose will be determined by the route, timing and frequency of administration as well as the existence, nature and extent of any adverse side effects that might accompany the administration of a particular therapeutic composition and the desired physiological effect. It is also known that various conditions or disease states, in particular, chronic conditions or disease states, may require prolonged treatment involving multiple administrations.

Therefore, the amount of the therapeutic composition must be effective to achieve an enhanced therapeutic index. If multiple doses are employed, the frequency of administration will depend, for example, on the type of subject. One skilled in the art can ascertain upon routine experimentation the appropriate route and frequency of administration in a given subject that are most effective in any particular case. Suitable doses and dosage regimens can be determined by conventionally known range-finding techniques. Generally, treatment is initiated with smaller dosages, which are less than the optimal dose of the compound. Thereafter, the dosage is increased by small increments until the optimal effect under the circumstances is obtained.

The therapeutic compositions for use in embodiments of the invention generally include carriers. These carriers may be any of those conventionally used and are limited only by the route of administration and chemical and physical considerations, such as solubility and reactivity with the therapeutic agent. In addition, the therapeutic composition may be formulated as polymeric compositions, inclusion complexes, such as cyclodextrin inclusion complexes, liposomes, microspheres, microcapsules, and the like, without limitation.

The pharmaceutically acceptable excipients described herein, for example, vehicles, adjuvants, carriers, or diluents, are well known and readily available. It is preferred that the pharmaceutically acceptable carrier be one which is chemically inert with respect to the therapeutic composition and one that has no detrimental side effects or toxicity under the conditions of use.

The choice of excipient will be determined, in part, by the particular therapeutic composition, as well as by the particular method used to administer the composition. Accordingly, there are a wide variety of suitable formulations of the pharmaceutical composition used in the embodiments of the invention. For example, the non-limiting formulations can be injectable formulations such as, but not limited to, those for intravenous, subcutaneous, intramuscular, intraperitoneal injection, and the like, and oral formulations such as, but not limited to, liquid solutions, including suspensions and emulsions, capsules, sachets, tablets, lozenges, and the like. Non-limiting formulations suitable for parenteral administration include aqueous and non-aqueous isotonic sterile injection solutions, including non-active ingredients such as antioxidants, buffers, bacteriostats, solubilizers, thickening agents, stabilizers, preservatives, surfactants, and the like. The solutions can include oils, fatty acids, including detergents and the like, as well as other well known and common ingredients in such compositions, without limitation.

Formulations described herein can be effective against a diverse fungal and bacterial drug-resistant microbial strains, such as Candida species (e.g., C. albicans and C. auris), gram-positive bacteria (e.g., MRSA and other staphylococci, streptococci, and enterococci), gram-negative bacteria (e.g., coliforms and Salmonella species).

1. A method for synthesizing bismuth nanoparticles comprising:
precipitating Bi(NO₃)₃ from an alkaline Bi(NO₃)₃ solution comprising chelator and polyvinylpyrrolidone (PVP) at a temperature of 60 to 75° C. by adding a reducing agent dropwise forming PVP coated BiNPs (PVP-BiNPs).

2. The method of claim 1, wherein the chelator is dimercaptopropanol (BAL).

3. The method of claim 1 or 2, wherein the reducing agent is NaBH₄.

4. The method of any one of claims 1 to 3, wherein the PVP has an average molecular weight of 5 to 20,000.

5. The method of any one of claims 1 to 4, wherein the PVP is PVP has an average molecular weight of 10,000.

6. The method of any one of claims 1 to 5, further comprising washing the PVP-BiNPs.

7. The method of claim 6, wherein the washing comprises pelleting the PVP-BiNPs, removing the supernatant, and washing the pellet with water.

8. The method of claim 7, wherein the PVP-BiNPs are pelleted by centrifugation at 3500 to 5000 g.

9. The method of claim 6, further comprising drying the washed PVP-BiNPs forming a dry powder.

10. The method of claim 9, further comprising storing the dried powder PVP-BiNPs at a temperature of 4° C. or lower.

11. The method of claim 9, further comprising storing the dried powder PVP-BiNPs protected from light.

12. A coated bismuth nanoparticle produced by the method of any one of claims 1 to 11.

13. A method of ameliorating bacterial growth comprising administering an effective amount of a bismuth nanoparticle produced by the method of any one of claims 1 to 12.
